(19) United States
(12) Patent Application Publication
(10) Pub. No.: US 2019/0231223 A1
(43) Pub. Date: Aug. 1, 2019

(54) METHOD, SYSTEM, PROGRAM, AND COMPUTER APPARATUS, FOR IDENTIFIYING SOURCE BODY REGION OF COMPENSATORY MOVEMENT, AND METHOD AND SYSTEM, FOR ELIMINATING COMPENSATORY MOVEMENT

(71) Applicant: Rika TAKAGI, Aichi (JP)

(72) Inventors: Rika TAKAGI, Aichi (JP); Hisayoshi YOSHITAKE, Aichi (JP); Toshimitsu ISHIZUKA, Kanagawa (JP)

(73) Assignee: Rika TAKAGI, Aichi (JP)

(21) Appl. No.: 16/339,560

(22) PCT Filed: Oct. 4, 2017
(86) PCT No.: PCT/JP2017/036090

§ 371 Date: Apr. 4, 2019

(30) Foreign Application Priority Data
Oct. 6, 2016 (JP) 2016-198454

(51) Int. Cl. A61B 5/107; A61B 5/11; G06K 9/00

(52) U.S. Cl. CPC A61B 5/1071; G06K 9/00342; A61B 5/1107

(57) ABSTRACT

Provided is a compensatory movement source body region identification method including: a step of first determination that determines existence of the compensatory movement in a first motion based on whether or not first measurement data satisfies a first criterion; and/or a step of second determination that determines existence of the compensatory movement in a second motion that is of lower complicity than the first motion based on whether or not second measurement data satisfies a second criterion; and a step of identifying at least one or more body region to be the source for causing the compensatory movement based on whether or not third measurement data in a third motion that is of lower complicity than the second motion satisfies a third criterion, when determined in the step of first determination and/or the step of second determination that there is the compensatory movement.

### TECHNICAL FIELD

The present invention relates to a method, a system, a program, and a computer apparatus for identifying a source body region of a compensatory movement by using data related to a still state or a moving state of a prescribed body region of an exerciser measured by one or more measurement device, and to a method and a system for eliminating the compensatory movement.

### BACKGROUND ART

In order to keep winning among the world's top athletes, it is necessary to improve the performance (stronger, faster, higher, etc.) while maintaining and improving the quality of motions, and thus, there is a demand for coaches and trainers capable of evaluating the motions of athletes and providing coaching, conditioning, training, treatment, and the like for improving the quality of motions. However, the evaluation method and the approaching method depend greatly on intuitions of the coaches and the trainers, so that there is no consistency.

Also, in spite of the fact that issues of unidentified complaints of sports enthusiasts and common people having no habit of exercising are started from a bad posture for a long period of time in an ordinal daily life activities or at work or from a custom forced to be in a specific posture (e.g., work forced to keep a same posture over a long period of time), for example, there is no attempt to improve the motions, postures, and postural habits, until pains and discomfort become tangible. In order to cut medical costs spent for unidentified complaints and to decrease economic loss, improvement and consciousness about the motions, postures, and postural habits as well as habituation of proper motions and the like are indispensable.

Recently, training for strengthening the trunk has been attracting attention for improving the quality of motions and maintaining health. For example, Patent Literature 1 proposes a system that discriminates existence of contraction of the trunk muscle under a prescribed movement by a sensor and, when there is no contraction of the trunk muscle, informs an exerciser that there is no contraction of the trunk muscle.

### CITATION LIST

### Patent Literature

Patent Literature 1: National Publication of International Patent Application No. 2016-504110

### SUMMARY OF INVENTION

### Technical Problem

However, the system described in Patent Literature 1 is for determining only the existence of contraction of the trunk muscle and unable to decide whether or not the trunk and other muscles are properly used for exercising. In order to improve the quality of motions and maintaining the health, it is insufficient to simply use the trunk alone and it is important to do exercise by properly using the trunk and other muscles. For example, when a movement using the muscle that is not supposed to be used instead of the muscle supposed to be used naturally or a movement imposing an excessive stress (load) on a certain joint or soft tissues of periarticular (also referred to as “compensatory movement” or “compensatory motion” hereinafter) is continued, the quality of motions is hard to be improved and issues of physical defects or unidentified complaints may arise.

The present invention is designed in view of above-described issues. That is, it is an object of the present invention to provide a consistent evaluation method for evaluating whether or not the trunk is used properly or whether or not there is a compensatory movement.

### Solution to Problem

The gists of the present invention are as follows.

[1] A compensatory movement source body region identification method for identifying a body region to be a source for causing a compensatory movement by using data regarding a still state or a moving state in a prescribed body region of an exerciser measured by one or more measurement device, the method comprising: a step of first determination that determines existence of the compensatory movement in a first motion based on whether or not first measurement data regarding the still state or the moving state of a first measurement body region group of the exerciser in the first motion satisfies a first criterion; and/or a step of second determination that determines existence of the compensatory movement in a second motion that is of lower complicity than the first motion based on whether or not second measurement data regarding the still state or the moving state of a second measurement body region group of the exerciser in the second motion satisfies a second criterion; and a step of identifying at least one or more body region to be the source for causing the compensatory movement based on whether or not third measurement data regarding the still state or the moving state of a third measurement body region group of the exerciser in a third motion that is of lower complicity than the second motion satisfies a third criterion, when determined in the step of first determination and/or the step of second determination that there is the compensatory movement.

[2] The compensatory movement source body region identification method according to [1], wherein the step of identifying the source body region further identifies a characteristic of the compensatory movement and a proportion contributing to occurrence of the compensatory movement in each of the source body regions.

[3] The compensatory movement source body region identification method according to [1] or [2], further comprising a step of inferring at least one or more body region to be the source for causing the compensatory movement based on whether or not the first measurement data satisfies the first criterion and/or the second measurement data satisfies the second criterion.

[4] The compensatory movement source body region identification method according to [3], wherein a type of the third motion is decided according to the source inferred to cause the compensatory movement in the step of inferring the source body region.

[5] The compensatory movement source body region identification method according to any one of [1] to [4], wherein the step of second determination is executed when determined in the step of first determination that there is the compensatory movement.

[6] The compensatory movement source body region identification method according to any one of [1] to [5], wherein the measurement device is one kind or more selected from a group consisting of a motion capture, a pressure sensor, an electromyogram meter, an ultrasonic measurement device, and a goniometer.

[7] A compensatory movement elimination method for eliminating a compensatory movement, comprising: identifying a body region to be a source for causing the compensatory movement by using the compensatory movement source body region identification method according to any one of [1] to [6]; and having the exerciser do a prescribed exercise depending on the identified body region.

[8] A compensatory movement source body region identification system for identifying a body region to be a source for causing a compensatory movement implemented by one or more measurement device measuring data regarding a still state or a moving state in a prescribed body region of an exerciser and a computer apparatus, the system comprising: a first determinator determining existence of the compensatory movement in a first motion based on whether or not first measurement data regarding the still state or the moving state of a first measurement body region group of the exerciser in the first motion satisfies a first criterion; and/or a second determinator determining existence of the compensatory movement in a second motion that is of lower complicity than the first motion based on whether or not second measurement data regarding the still state or the moving state of a second measurement body region group of the exerciser in the second motion satisfies a second criterion; and a source body region identifier identifying at least one or more body region to be the source for causing the compensatory movement based on whether or not third measurement data regarding the still state or the moving state of a third measurement body region group of the exerciser in a third motion that is of lower complicity than the second motion satisfies a third criterion, when determined by the first determiner and/or the second determiner that there is the compensatory movement.

[9] The compensatory movement source body region identification system according to [8], wherein the source body region identifier further identifies a characteristic of the compensatory movement and a proportion contributing to occurrence of the compensatory movement in each of the source body regions.

[10] The compensatory movement source body region identification system according to [8] or [9], further comprising a source body region inferencer inferring at least one or more body region to be the source for causing the compensatory movement based on whether or not the first measurement data satisfies the first criterion and/or the second measurement data satisfies the second criterion.

[11] The compensatory movement source body region identification system according to [10], wherein a type of the third motion is decided according to the source inferred to cause the compensatory movement by the source body region inferencer.

[12] The compensatory movement source body region identification system according to any one of [8] to [111], wherein existence of the compensatory movement in the second motion is determined by the second determinator when determined by the first determinator that there is the compensatory movement.

[13] The compensatory movement source body region identification system according to any one of [8] to [12], wherein the measurement device is one kind or more selected from a group consisting of a motion capture, a pressure sensor, an electromyogram meter, an ultrasonic measurement device, and a goniometer.

[14] A compensatory movement elimination system eliminating a compensatory movement through identifying a body region to be a source for causing the compensatory movement by using the compensatory movement source body region identification system according to any one of [8] to [13]; and having the exerciser do a prescribed exercise depending on the identified body region.

[15] A program causing a computer apparatus to identify a body region to be a source for causing a compensatory movement by using data regarding a still state or a moving state in a prescribed body region of an exerciser measured by one or more measurement device, the program causing the computer apparatus to function as: a first determinator determining existence of the compensatory movement in a first motion based on whether or not first measurement data regarding the still state or the moving state of a first measurement body region group of the exerciser in the first motion satisfies a first criterion; and/or a second determinator determining existence of the compensatory movement in a second motion that is of lower complicity than the first motion based on whether or not second measurement data regarding the still state or the moving state of a second measurement body region group of the exerciser in the second motion satisfies a second criterion; and a source body region identifier identifying at least one or more body region to be the source for causing the compensatory movement based on whether or not third measurement data regarding the still state or the moving state of a third measurement body region group of the exerciser in a third motion that is of lower complicity than the second motion satisfies a third criterion, when determined by the first determiner and/or the second determiner that there is the compensatory movement.

[16] A computer apparatus identifying a body region to be a source for causing a compensatory movement by using data regarding a still state or a moving state in a prescribed body region of an exerciser measured by one or more measurement device, the computer apparatus comprising: a first determinator determining existence of the compensatory movement in a first motion based on whether or not first measurement data regarding the still state or the moving state of a first measurement body region group of the exerciser in the first motion satisfies a first criterion; and/or a second determinator determining existence of the compensatory movement in a second motion that is of lower complicity than the first motion based on whether or not second measurement data regarding the still state or the moving state of a second measurement body region group of the exerciser in the second motion satisfies a second criterion; and a source body region identifier identifying at least one or more body region to be the source for causing the compensatory movement based on whether or not third measurement data regarding the still state or the moving state of a third measurement body region group of the exerciser in a third motion that is of lower complicity than the second motion satisfies a third criterion, when determined by the first determiner and/or the second determiner that there is the compensatory movement.

[17] A compensatory movement source body region identification method for identifying a body region to be a source for causing a compensatory movement based on a still state or a moving state in a prescribed body region of an exerciser, the method comprising: a step of first determination that determines existence of the compensatory movement in a first motion based on whether or not the still state or the moving state of a first measurement body region group of the exerciser in the first motion satisfies a first criterion; and/or a step of second determination that determines existence of the compensatory movement in a second motion that is of lower complicity than the first motion based on whether or not the still state or the moving state of a second measurement body region group of the exerciser in the second motion satisfies a second criterion; and a step of identifying at least one or more body region to be the source for causing the compensatory movement based on whether or not the still state or the moving state of a third measurement body region group of the exerciser in a third motion that is of lower complicity than the second motion satisfies a third criterion, when determined in the step of first determination and/or the step of second determination that there is the compensatory movement.

[18] A source body region identification system for executing a compensatory movement source body region identification method that identifies a body region to be a source for causing a compensatory movement based on a still state or a moving state in a prescribed body region of an exerciser, wherein: the compensatory movement source body region identification method comprises a step of first determination that determines existence of the compensatory movement in a first motion based on whether or not the still state or the moving state of a first measurement body region group of the exerciser in the first motion satisfies a first criterion and/or a step of second determination that determines existence of the compensatory movement in a second motion that is of lower complicity than the first motion based on whether or not the still state or the moving state of a second measurement body region group of the exerciser in the second motion satisfies a second criterion, and a step of identifying at least one or more body region to be the source for causing the compensatory movement based on whether or not the still state or the moving state of a third measurement body region group of the exerciser in a third motion that is of lower complicity than the second motion satisfies a third criterion, when determined in the step of first determination and/or the step of second determination that there is the compensatory movement; and the source body region identification system comprises a first recording medium recording the first criterion for determining existence of the compensatory movement in the first motion for the first measurement body region group of each exerciser and/or a second recording medium recording the second criterion for determining existence of the compensatory movement in the second motion for the second measurement body region group of each exerciser, and a third recording medium recording the third criterion for identifying the body region to be the source for causing the compensatory movement based on the still state or the moving state in the third measurement body group of the exerciser.

### Advantageous Effects of Invention

With the present invention, it becomes possible to clarify the points to be focused (e.g., coaching point, conditioning method, body region to be trained, body region to receive treatment) in order to maintain and improve the motions through monitoring performance motions before the performance of the athlete becomes stagnant or before becoming poor condition and to plan a strategy to win. Further, through monitoring the ordinal daily life activities of sports enthusiasts and common people having no habit of doing exercise, it becomes possible to improve the motions and postures before the issues of unidentified complaints become tangible.

### BRIEF DESCRIPTION OF DRAWINGS

FIG. 1 is a block diagram illustrating a configuration of the computer apparatus corresponding to at least one of the first embodiment of the present invention.

FIG. 2 is a flowchart of the program execution processing corresponding to at least one of the first embodiment of the present invention.

FIG. 3 is a flowchart of the program execution processing corresponding to at least one of the first embodiment of the present invention.

FIG. 4 is a schematic view illustrating an example of a block forming method when an exerciser is in a standing position, corresponding to at least one of the first embodiment of the present invention.

### DESCRIPTION OF EMBODIMENTS

While embodiments of the present invention will be described hereinafter by referring to the accompanying drawings and the like, it is to be noted that the present invention is not limited to the following embodiments without departing from the spirit of the present invention. Referring to reference numerals applied to each region of a human body in the drawings, different reference numerals may be applied in some cases even for the same body region. Further, orders of each processing forming flowcharts described hereinafter are set in no specific order as long as there is no confliction and inconsistency generated in the content of the processing.

### First Embodiment

First, outline of a first embodiment of the present invention will be described. Hereinafter, the first embodiment will be described by referring to a compensatory movement source body region identification system that identifies the body region as a source for causing a compensatory movement implemented by one or more measurement device measuring (also referred to as “monitoring”) data regarding a still state or a moving state of a prescribed body region of an exerciser and by a computer apparatus.

In the present description, “first motion”, “second motion”, and “third motion” include not only exercising by moving bodies but also taking a prescribed posture and maintaining a still state. “To determine existence of the compensatory movement” means to decide whether or not the exerciser is making a movement using the muscle that is not supposed to be used instead of the muscle supposed to be used naturally or making a movement imposing an excessive stress (load) on a certain joint or soft tissues of periarticular; in other words, it is to decide whether or not movement the exerciser is doing is deviated from an ideal (normal) motion/movement pattern for the exerciser.

(Measurement Device)

There is no specific limit set for the measurement device as long as the device is capable of measuring the data regarding a still state or a moving state in a prescribed body region of the exerciser, and for example, it is possible to use a motion capture, a pressure sensor, an electromyogram meter, an ultrasonic measurement device, or a goniometer as appropriate according to the motions to be exercised by the exerciser. In respect that it is possible to acquire measurement data without a coach or a trainer or to acquire accurate measurement data, the motion capture, the pressure sensor, the electromyogram meter, the ultrasonic measurement device, for example, is preferable. Note that a plurality of one or more types of measurement devices may be used in combination.

As the motion capture, it is possible to employ any type such as an inertial sensor type, an optical type, a mechanical type, a magnetic type, or a video type as appropriate. When attaching a sensor or a marker (referred to as “sensor or the like” hereinafter) to the exerciser, the attaching position or the like may be determined as appropriate depending on the type of exercise the exerciser is about to do or the body region to be monitored. For example, the sensor or the like may be attached to both ears, the glabella, or the like when monitoring motions of the head, may be attached to each of acromial lateral margins or lateral humeral epicondyle lateral margins on both sides when monitoring motions of the glenohumeral joint, may be attached to spinous process of the second thoracic vertebra, spinous process of the seventh thoracic vertebra, each of acromial superior margins, spina scapulae, angulus superior scapula, angulus inferior scapulae, medials margin of scapulae, or the like on both sides when monitoring motions of the scapulae, may be attached to manubrium sterni jugular notch, ensiform process, spinous process of the seventh cervical vertebra, spinous process of the first to tenth thoracic vertebra or the like, when monitoring motions of the thorax region, may be attached to each of anterior superior iliac spines, posterior superior iliac spines, pubic tubercles, ischia or the like on both sides, when monitoring motions of the pelvic region, may be attached to, in addition to the regions for monitoring the pelvic region, greater trochanter lateral region for grasping the positions of the femurs on both sides and inferior margin of femur lateral condyle or the like, when monitoring motions of the coxae region, may be attached to the greater trochanter lateral region or the inferior margin of femur lateral condyle for grasping the positions of each of femurs on both sides, and tuberositas tibiae, fibular head lateral margin, medial margin of medial malleolus, lateral margin of lateral malleolus, or the like for grasping the tibia and fibula when monitoring motions of the knee joints, and may be attached to each instep side of the first to fifth metatarsal bone distal portions, posterior calcaneal process of cuboids, medial margins of medial malleoli, lateral margins of lateral malleoli, or the like on both sides, when monitoring motions of foot regions. The sensor or the like may be attached to at least one or more point mentioned above, but, in terms of improving the accuracy of monitoring, it is preferable to attach the sensor or the like to a plurality of points. The sensor or the like is not limited to be attached only to the points mentioned above but may also be attached to other points.

It is preferable for the data acquired by monitoring to be transmitted from the measurement device to a computer apparatus through wired or wireless means. It is also possible to employ a configuration allowing the data acquired by a coach, a trainer, an exerciser, or the like to be inputted directly to the computer apparatus.

(Computer Apparatus)

Examples of the computer apparatus may be a personal computer, a smartphone, a tablet terminal, a mobile terminal, a PDA, a wearable terminal, and a server device, but the computer apparatus is not limited to those as long as the device has a processing capacity capable of identifying the source body region of the compensatory movement based on the data measured by the measurement device.

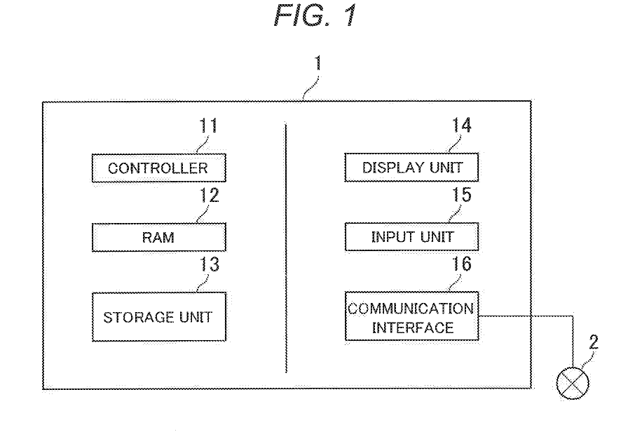

FIG. 1 is a block diagram illustrating a configuration of the computer apparatus corresponding to at least one of the first embodiment of the present invention. A computer apparatus 1 includes at least a controller 11, a RAM (Random Access Memory) 12, a storage unit 13, a display unit 14, an input unit 15, and a communication interface 16, and each of those are connected via an internal bus.

The controller 11 is formed with a CPU (Central Processing Unit) and a ROM (Read Only Memory), and executes programs stored in the storage unit 13 to control the computer apparatus 1. Further, the controller 11 includes an internal timer for clocking the time. The RAM 12 is a work area of the controller 11. The storage unit 13 is a memory area for storing the programs and data.

The communication interface 16 is capable of connecting to a communication network 2 by wireless or wired means, and capable of receiving data from the measurement device via the communication network 2. The data received via the communication interface 16 is loaded on the RAM 12 and calculation processing is performed by the controller 11.

The display unit 14 has a display screen for displaying images, and displays the images on the display screen based on video signals outputted from the controller 11. The input unit 15 is formed with a mouse, a keyboard, or a touch panel, for example. When employing the touch panel as the input unit 15, the touch panel can also function as the display screen of the display unit 14. Input information inputted to the input unit 15 is stored in the RAM 12, and the controller 11 executes various types of calculation processing based on the input information.

Note that the computer apparatus 1 may also include a sound output unit connected to a sound output device (e.g., speaker), for example, other than the above-described components. When the controller 11 outputs an instruction to output sound to a sound processor, the sound processor outputs a sound signal to the sound output device. It is preferable for the sound output device to output an instruction regarding exercise content and feedback for the exercise, for example, by voice.

Next, functions of the computer apparatus 1 will be described. The computer apparatus 1 has a specific-movement-or-the-like instruction function, a monitoring information reception function, a specific-movement-or-the-like-data determining function, a coaching function, a basic movement instruction function, a basic movement data determining function, a source body region inferring function, a separate-movement-or-the-like determination function, a separate-movement-or-the-like instruction function, a source body region identification function, and a function-improving-exercise instruction function, for example.

The specific-movement-or-the-like instruction function has a function of giving an instruction to the exerciser via the display screen, sound, or the like in regards to types of specific movement or daily movement (also referred to as “specific movement or the like” hereinafter) the exerciser is to do as a first motion. Note that “specific movement” herein means each movement peculiar to respective types of sports and the like, and means movements with toughness and speeds. Examples may be a swing motion in case of hammer throw, and a pitching motion and a batting motion in case of baseball. Further, as “specific movement”, movements used for measuring the exercise capacity, such as standing broad jump, vertical jump, single-leg three-step test, and the like are also included as long as the movements need the toughness and speeds. “Daily movement” means basic daily-life motions repeated in daily life, and examples may be a walking motion, a motion changing from spine position or a sitting position to a standing position.

The types of specific movement or the like to be done by the exerciser may be determined by the computer apparatus based on the information of the exerciser inputted via the input unit 15 or may be determined by the exerciser oneself through displaying choices on the display screen, for example, or, alternatively, a specific movement (e.g., walking motion) may be set in advance to be instructed.

The monitoring information reception function has a function of receiving, from the measurement device, first measurement data related to a still state or a moving state of a prescribed body region of the exerciser monitored by the measurement device regarding the specific movement or the like of the exerciser, the basic movement to be described later, and the separate movement or static posture upkeep (also referred to as “separate movement or the like” hereinafter).

The specific-movement-or-the-like-data determining function has a function of determining existence of the compensatory movement in the specific movement or the like based on whether or not the first measurement data regarding the specific movement or the like received by the monitoring information reception function satisfies a first criterion. The first criterion will be described in detail in a latter paragraph.

When there is a compensatory movement in the specific movement or the like and the basic movement to be described later, the coaching function has a function of presenting a coaching program for eliminating the compensatory movement. The coaching program is a program presenting how the specific movement or the like and the basic movement the exerciser did was different from the motions considered ideal or a program giving an advice for becoming closer to the movement considered ideal or giving a practice assignment. Specifically, an example may be a package of at least one or more contents selected from sharing of visualized ideal movements, instructions by the voice outputted from the sound output unit or by encouragements from the coach or the like, presenting points to be conscious about, partial practicing of the motions configuring the specific movement or the like and the basic movement.

It is preferable for the content of the coaching program to be determined according to a degree of the data acquired by monitoring unsatisfying the prescribed criterion or according to the compensatory movement patterns, for example. The coaching program may be displayed on the display screen or may be outputted from a printer or the like connected via the communication interface 16.

The basic movement instruction function has a function of giving an instruction to the exerciser via the display screen, sound, or the like in regards to types of the basic movement the exerciser is supposed to do as a second motion. Note here that “basic movement” means multi-joint exercise and the like that are less complicated than the specific movement or the like and has no shift in the base of support, and examples thereof may be a squat motion (overhead squat or the like), a one-leg standing motion (one-leg squat), a lunge motion (lunge-stance rotational throw or the like), a forward bending motion, an extension motion, side bending motion, a rotation motion, an upper limb mobility test, and the like.

The types of the basic movement for encouraging the exerciser to do may be determined by the computer apparatus based on the information of the exerciser inputted via the input unit 15 and the first measurement data and the like regarding the specific movement or the like, may be determined by the exerciser oneself through displaying choices on the display screen, for example, or may give an instruction in advance to do a specific movement (e.g., overhead squat).

The basic movement data determining function has a function of determining existence of the compensatory movement in the basic movement based on whether or not second measurement data regarding the basic movement received by the monitoring information reception function satisfies a second criterion. The second criterion will be described in detail in a latter paragraph.

The source body region inferring function has a function of inferring at least one or more body region as the source for causing the compensatory movement based on whether or not the first measurement data satisfied the first criterion and/or based on whether or not the second measurement data satisfies the second criterion. Having the source body region inferring function makes it possible to provide a more proper coaching program or a function improving exercise program even at a stage where the source body region has not been identified when the first measurement data does not satisfy the first criterion or when second measurement data does not satisfy the second criterion.

The separate movement or the like determination function has a function of determining types of the separate movement or the like or the static posture the exerciser is supposed to take as a third motion based on the data and the like regarding the specific movement or the like and the basic movement or the inferred result acquired by the source body region inferring function. Note here that “separate movement” and “static posture” are movement and posture upkeep for evaluating mobility and stability of a single joint with less complicity than the basic movement. Further, separate movement may include not only the movement done by the exerciser alone but also an evaluation of range of motion of a body region passively held and moved by a coach or trainer as well as muscular strength measurement. The separate-movement-or-the-like instruction function has a function of giving an instruction to the exerciser via the display screen or the like to do the separate movement or the like determined by the separate movement or the like determination function.

The source body region identification function has a function of identifying at least one or more body region as the source for causing the compensatory movement in the specific movement or the like and/or the basic movement based on whether or not third measurement data regarding the separate movement or the like received by the monitoring reception function satisfies a third criterion. The third criterion will be described in detail in a latter paragraph.

Further, it is preferable for the source body region identification function to have a function of identifying a proportion contributing to characteristic of the compensatory movement and occurrence of the compensatory movement in each source body region based on whether or not the third measurement data satisfies the third criterion. Through identifying the proportion contributing to the characteristic of the compensatory movement and occurrence of the compensatory movement in each source body region, it becomes possible to place priority on each body region that needs improvement in the function improving exercise program to be described later so that a proper function improving exercise program can be provided.

The function-improving-exercise instruction function has a function of giving an instruction to do the function improving exercise program for eliminating the compensatory movement or for maintaining the state where the compensatory movement is being eliminated when the compensatory movement is eliminated by the coaching function, when the body region as the source for causing the compensatory movement or the characteristic are inferred by the source body region inferring function, or when the body region as the source for causing the compensatory movement is identified by the source body region identification function. The content of the function improving exercise program can be determined as appropriate depending on the degree of the data regarding the specific movement or the like and the basic movement unsatisfying the prescribed criterion, depending on the compensatory movement pattern, or depending on the source body region of the compensatory movement inferred or identified. In terms of providing a more effective exercise program by eliminating the compensatory movement, it is preferable to determine the content of the function improving exercise program by taking in account not only the source body region of the compensatory movement but also the characteristic of the compensatory movement and the proportion contributing to occurrence of the compensatory movement in each source body region. The function improving exercise program may be displayed on the display screen or may be outputted from a printer or the like connected via the communication interface 16.

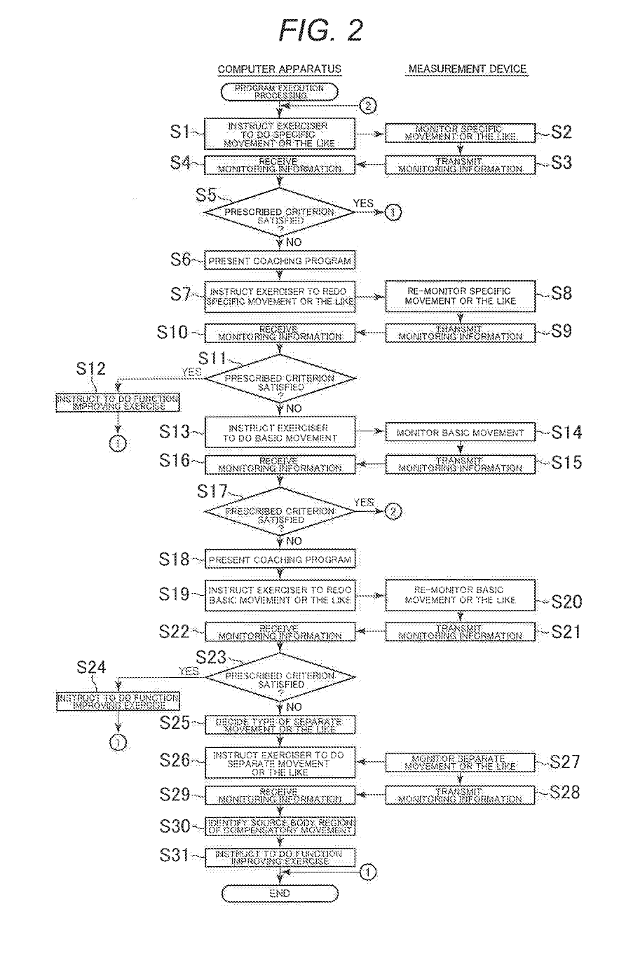

Next, program execution processing for identifying the source body region of the compensatory movement corresponding to at least one of the first embodiment will be described. FIG. 2 is a flowchart of the program execution processing corresponding to at least one of the first embodiment of the present invention.

First, the computer apparatus 1 gives an instruction to the exerciser to do the specific movement or the like (step S1). Then, the specific movement or the like being conducted by the exerciser are monitored by the measurement device (step S2), and the measurement data regarding the specific movement or the like acquired by the monitoring is transmitted to the computer apparatus 1 from the measurement device (step S3).

The computer apparatus 1 receives the measurement data regarding the specific movement or the like from the measurement device (step S4), and determines whether or not the measurement data satisfies the first criterion (step S5). When the measurement data satisfies the first criterion (Yes in step S5), an ideal motion (which may be a motion of the exerciser determined to satisfy the first criterion) with no compensatory movement in the specific movement or the like, for example, is visualized and displayed on the display device, a program for increasing the exercise amount in a daily life and improving the athletic ability is presented in accordance with an objective and a motive of the exerciser, an advice for conducting regular monitoring or the like is displayed, and the processing is terminated.

Now, the first criterion will be described. With the first criterion, existence of the compensatory movement in the specific movement or the like is determined from following four points of view, for example. The first point of view is whether or not motion of the center axis (spinal column) of the trunk is normal. For the spinal column, for example, conducted are an evaluation regarding whether or not the spinal column is perpendicular with respect to the pelvis or a straight line connecting coxae on left and right and an evaluation regarding whether or not there is unnecessary or excessive side bending, rotation, flexion, or extension of the spinal column in the motion in any types of postures taken in the motions. The second point of view is whether or not a kinematic chain is normal. For the kinematic chain, for example, conducted are an evaluation regarding whether or not each one of five rotation axes is rotated properly, an evaluation regarding whether or not the rotation axes do not move simultaneously but move in conjunction with each other in a linked manner, and an evaluation regarding whether or not rotation starts from the rotation axis appropriate for the motion.

The third point of view is whether or not shift in the center-of-mass is done appropriately. For the shift in the center-of-mass, for example, conducted are an evaluation regarding whether or not a correlation regarding three center-of-mass points that are the center-of-mass point of a whole body, the center-of-mass point of an upper half body, and the center-of-mass point of a head is appropriate, an evaluation regarding whether or not a correlation between the base of support and the center-of-mass line is appropriate, and an evaluation regarding whether or not shift in the center-of-mass (shift from where to where) in that motion is appropriate. The fourth view of point is whether or not interrelation of the upper limbs and lower limbs is appropriate. For the interrelation of the upper limbs, for example, conducted are an evaluation regarding whether or not timings and proportions of joint torques of respective motions of the wrists, elbows, and shoulders (the joint torque of which body region is high) in the motion are appropriate, and an evaluation regarding whether or not positional relationships between the upper limbs and the pelvis or the chest/shoulder girdle are appropriate. For the interrelation of the lower limbs, for example, conducted is an evaluation regarding whether or not timings and proportions of joint torques of respective motions of ankles, knee joints, and coxae (the joint torque of which body region is high) in the motion are appropriate.

When monitoring the specific movement or the like, it is preferable to use the measurement device(s) capable of conducting evaluations based on the four points of view described above. For example, it is preferable to attach a sensor or the like at a position capable of conducting the evaluations based on the four points of view described above.

Now, steps S1 to S5 will be described in more detail by referring to a case where a walking motion is done as the specific movement or the like and a motion capture is used as the measurement device. First, the exerciser places a sensor or the like directly on the body of oneself or on the worn clothes.

As the attaching positions of the sensor or the like, the positions capable of acquiring following body region information are preferable. In order to detect the compensatory movement occurring in the periphery of feet and foot joints, it is preferable to be able to acquire body region information of the second toe, third toe, and calcaneus bone of each of both feet, for example. Further, in order to detect the compensatory movement occurring in periphery of the knee joints, it is preferable to be able to acquire body region information of a frontal plane in the longitudinal axis of the femur, a frontal plane in the longitudinal axis of the tibia, a sagittal plane in the longitudinal axis of the femur, and a sagittal plane in the longitudinal axis of the tibia, for example. Also, in order to detect the compensatory movement occurring in periphery of the coxae, it is preferable to be able to acquire body region information of the torso perpendicular line, the pelvic region, and the longitudinal axis of the femur, for example. In order to detect the compensatory movement occurring in periphery of the pelvis and the lumber region, it is preferable to be able to acquire body region information of the anterior superior iliac spines on left and right and the posterior superior iliac spine on left and right, for example. Further, in order to detect the compensatory movement occurring in periphery of the thorax region, shoulder region, neck region, and head, it is preferable to be able to acquire body region information of acromioclavicular joints on left and right, near the external acoustic openings on left and right, and the parietal region of the head, for example.

As the specific attaching position of the sensor or the like, following positions are preferable. In order to detect the compensatory movement occurring in the periphery of feet and foot joints, each of the instep side of the first to fifth metatarsal bone distal portions, posterior calcaneal process of cuboids, the medial margins of medial malleoli, or the lateral margins of lateral malleoli on both sides is preferable, for example. In order to detect the compensatory movement occurring in the periphery of the knee joints, each of the greater trochanter lateral regions or the inferior margins of femur lateral condyles for grasping the positions of the femurs on both sides and the tuberositas tibiae, the lateral margin of the head of fibula, the medial margin of medial malleolus or the lateral margin of lateral malleolus for grasping the tibia and fibula are preferable, for example. In order to detect the compensatory movement occurring in the periphery of coxae, each of the anterior superior iliac spines, the posterior superior iliac spines, the pubic tubercles, or the ischia on both sides for grasping the motion of the pelvic region and, in addition, the greater trochanter lateral regions or the inferior margins of femur lateral condyles for grasping the positions of each of the femurs on both side are preferable, for example. In order to detect the compensatory movement occurring in the periphery of the pelvis and the limber region, each of the anterior superior iliac spines, the posterior anterior iliac spins, the pubic tubercles, or the ischia on both sides is preferable, for example. In order to detect the compensatory movement occurring in the periphery of the shoulder girdle, neck region, and head, each of the acromial lateral margins and the lateral humeral epicondyle lateral margins on both sides, the spinous process of second thoracic vertebra, the spinous process of seventh thoracic vertebra, each of acromial superior margins, spina scapulae, angulus superior scapulae, angulus inferior scapulae, and medial margins of scapulae on both sides, the manubrium sterni jugular notch, both ears, or glabella are preferable, for example. While it is fine to attach the sensor or the like at least one or more point mentioned above, it is preferable to attach the sensor or the like at a plurality of points in terms of improving the accuracy of monitoring. Note that the attaching points of the sensor or the like are not limited to the points mentioned above, and other points may be selected for attachment.

While examples of the first criterion in a case where the specific movement or the like is a walking motion are presented in TABLE 1, the first criterion are not limited only to those presented in TABLE 1. Note that “perpendicular line” in the present description means a line perpendicular to a floor surface. Further, “horizontal plane” means a plane horizontal to the floor surface. Also, “determination point” means a point to be focused when determining existence of the compensatory movement, and the sensor or the like is attached to the position capable of making determination at the determination point.

TABLE 1

| | COMPENSATORY | | |
|---|---|---|---|
| BODY REGION | MOVEMENT PATTERN | DETERMINATION POINT | NORMAL |
| FOOT REGION | TOE FACING | ANGLE BETWEEN TRAVEL DIRECTION | 0° OR MORE AND |
| AND FOOT | OUTWARD | AND LINE PASSING THROUGH MID | LESS THAN 13° |
| JOINT | | POINT OF SECOND TOE AND THIRD | |
| | | TOE AND HEEL | |
| | TOE FACING | ANGLE BETWEEN TRAVEL DIRECTION | 0° OR MORE AND |
| | INWARD | AND LINE CONNECTING MID | LESS THAN 13° |
| | | POINT OF SECOND TOE AND | |
| | | THIRD TOE AND HEEL | |
| | CIRCUMDUCTION OF | TRAVEL DIRECTION AND SECOND TOE | TIPTOES MOVING STRAIGHT |
| | TOE TIP | | FORWARD IN SWING PHASE |
| | DIFFERENCE IN LEFT | DISTANCE BETWEEN HEEL ON ONE | LESS THAN 2 CM IN |
| | AND RIGHT STRIDES | SIDE AND THE OTHER | DIFFERENCE BETWEEN LEFT |
| | | | AND RIGHT |
| | ARCH CRUSHED | ANGLE BETWEEN MAJOR AXIS OF | 5° OR MORE AND |
| | | TIBIA AND CALCANEUS BONE | LESS THAN 14° |
| | SUPINATION OF | ANGLE BETWEEN MAJOR AXIS OF | 5° OR MORE AND |
| | FOOT REGION | TIBIA AND CALCANEUS BONE | LESS THAN 14° |
| KNEE JOINT | KNEE IN | ANGLE BETWEEN MAJOR AXIS OF | 170° OR MORE AND |
| | | FEMUR IN FRONTAL PLANE AND | LESS THAN 180° |
| | | MAJOR AXIS OF TIBIA | |
| | KNEE OUT | ANGLE BETWEEN MAJOR AXIS OF | 170° OR MORE AND |
| | | FEMUR IN FRONTAL PLANE AND | LESS THAN 180° |
| | | MAJOR AXIS OF TIBIA | |
| | OVER FLEXION | ANGLE BETWEEN MAJOR AXIS OF | FROM 5° FLEXION |
| | | FEMUR IN SAGITTAL PLANE AND | POSITION TO |
| | | MAJOR AXIS OF TIBIA | 15° FLEXION POSITION |
| | OVER | ANGLE BETWEEN MAJOR AXIS OF | FROM 15° FLEXION |
| | EXTENSION | FEMUR IN SAGITTAL PLANE | POSITION TO |
| | | AND MAJOR AXIS OF TIBIA | 5° FLEXION POSITION |
| COXA | INSUFFICIENT | ANGLE BETWEEN BODY | FROM 20° FLEXION |
| | EXTENSION | PERPENDICULAR LINE AND | POSITION TO 16 TO |
| | | MAJOR AXIS OF FEMUR | 25° EXTENSION POSITION |
| | INSUFFICIENT | ANGLE BETWEEN BODY | FROM 20° EXTENSION POSITION |
| | FLEXION | PERPENDICULAR LINE AND | TO 25 TO 30° FLEXION POSITION |
| | | MAJOR AXIS OF FEMUR | IN MIDSWING PHASE |
| PELVIS AND | OVER ANTERIOR | ANGLE BETWEEN HORIZONTAL PLANE | 8° OR MORE AND |
| LUMBAR | TILT, SWAY BACK | PASSING THROUGH ANTERIOR SUPERIOR | LESS THAN 12° |
| REGION | | ILIAC SPINE AND LINE PASSING THROUGH | |
| | | ANTERIOR SUPERIOR ILIAC SPINE AND | |
| | | POSTERIOR SUPERIOR ILIAC SPINE | |
| | OVER POSTERIOR | ANGLE BETWEEN HORIZONTAL PLANE | 8° OR MORE AND |
| | TILT, FLEXION | PASSING THROUGH ANTERIOR SUPERIOR | LESS THAN 12° |
| | | ILIAC SPINE AND LINE PASSING THROUGH | |
| | | ANTERIOR SUPERIOR ILIAC SPINE AND | |
| | | POSTERIOR SUPERIOR ILIAC SPINE | |
| | OVER ROTATION | ANGLE BETWEEN FRONTAL PLANE | 8° OR MORE AND |
| | | AND LINE PASSING THROUGH LEFT | LESS THAN 12° |
| | | ANTERIOR SUPERIOR ILIAC SPINE | |
| | | AND RIGHT ANTERIOR SUPERIOR | |
| | | ILIAC SPINE | |
| | INSUFFICIENT | ANGLE BETWEEN FRONTAL PLANE | 8° OR MORE AND |
| | ROTATION | AND LINE PASSING THROUGH LEFT | LESS THAN 12° |
| | | ANTERIOR SUPERIOR ILIAC SPINE | |
| | | AND RIGHT ANTERIOR SUPERIOR | |
| | | ILIAC SPINE | |
| | OVER TRANSVERSE | ANGLE BETWEEN HORIZONTAL PLANE | LESS THAN 3° |
| | TILT OF PELVIS | AND LINE PASSING THROUGH LEFT | |
| | | POSTERIOR SUPERIOR ILIAC SPINE | |
| | | AND RIGHT POSTERIOR SUPERIOR | |
| | | ILIAC SPINE | |
| THORAX, | THORACIC | ANGLE BETWEEN HORIZONTAL PLANE | 5° OR MORE AND |
| SHOULDER, | KYPHOSIS | PASSING THROUGH MANUBRIUM STERNI | LESS THAN 15° |
| NECK AND | | JUGULAR NOTCH AND LINE CONNECTING | |
| HEAD REGIONS | | MANUBRIUM STERNI JUGULAR NOTCH AND | |
| | | SPINOUS PROCESS OF SEVENTH | |
| | | CERVICAL VERTEBRA | |
| | FOREHEAD | DISTANCE BETWEEN PERPENDICULAR LINE | LESS THAN 2.0 CM |
| | POSTURE | PASSING THROUGH ACROMIOCLAVICULAR | |
| | | JOINT AND PERPENDICULAR LINE PASSING | |
| | | THROUGH EXTERNAL ACOUSTIC OPENING | |
| | TRANSVERSE | SHIFT DISTANCE OF PARIETAL REGION TO | LESS THAN 2.5 CM |
| | OVERSHIFT OF | LEFT OR RIGHT WITH RESPECT TO | |
| | HEAD POSITION | PERPENDICULAR LINE PASSING THROUGH | |
| | | MANUBRIUM STERNI JUGULAR NOTCH | |

| | COMPENSATORY | | |
|---|---|---|---|
| BODY REGION | MOVEMENT PATTERN | WARNING | NEED CAUTION |
| FOOT REGION | TOE FACING | 13° OR MORE AND LESS THAN 20° | 20° OR MORE |
| AND FOOT | OUTWARD | | |
| JOINT | | | |
| | TOE FACING | −8° OR MORE AND LESS THAN 0° | LESS THAN −8° |
| | INWARD | | |
| | CIRCUMDUCTION OF | TIPTOES MOVING BY MAKING SMALL | TIPTOES MOVING BY MAKING |
| | TOE TIP | ARC IN SWING PHASE | LARGE ARC IN SWING PHASE |
| | DIFFERENCE IN LEFT | 2 CM OR MORE AND LESS THAN 5 CM | 5 CM OR MORE IN DIFFERENCE |
| | AND RIGHT STRIDES | IN DIFFERENCE BETWEEN LEFT AND | BETWEEN LEFT AND RIGHT |
| | | RIGHT | |
| | ARCH CRUSHED | 14° OR MORE AND LESS THAN 20° | 20° OR MORE |
| | SUPINATION OF | −3° OR MORE AND LESS THAN 5° | LESS THAN −3° |
| | FOOT REGION | | |
| KNEE JOINT | KNEE IN | 160° OR MORE AND LESS THAN 170° | LESS THAN 160° |
| | KNEE OUT | 180° OR MORE AND LESS THAN 180° | 190° OR MORE |
| | OVER FLEXION | FROM 5° FLEXION POSITION TO | SUDDEN FLEXION OF 20° OR |
| | | 20° FLEXION POSITION | MORE |
| | OVER EXTENSION | FROM 15° FLEXION POSITION TO | OVEREXTENSION OF 0° OR LESS |
| | | 0° FLEXION POSITION | |
| COXA | INSUFFICIENT | FROM 20° FLEXION POSITION TO 6 TO | FROM 20° FLEXION POSITION TO |
| | EXTENSION | 15° EXTENSION POSITION | EXTENSION POSITION OF 5° OR |
| | | | LESS |
| | INSUFFICIENT | FROM 20° EXTENSION POSITION TO | FROM 20° EXTENSION POSITION |
| | FLEXION | 20 TO 24° FLEXION POSITION IN | TO FLEXION POSITION OF LESS |
| | | MIDSWING PHASE | THAN 20° IN MIDSWING PHASE |
| PELVIS AND | OVER ANTERIOR | 12° OR MORE AND LESS THAN 15° | 15° OR MORE |
| LUMBAR | TILT, SWAY BACK | | |
| REGION | | | |
| | OVER POSTERIOR | 3° OR MORE AND LESS THAN 8° | LESS THAN 3° |
| | TILT, FLEXION | | |
| | OVER ROTATION | 12° OR MORE AND LESS THAN 15° | 15° OR MORE |
| | INSUFFICIENT | 4° OR MORE AND LESS THAN 8° | LESS THAN 4° |
| | ROTATION | | |
| | OVER TRANSVERSE | 3° OR MORE AND LESS THAN 7° | 7° OR MORE |
| | TILT OF PELVIS | | |
| THORAX, | THORACIC | 15° OR MORE AND LESS THAN 25° | 25° OR MORE |
| SHOULDER, | KYPHOSIS | | |
| NECK AND | FOREHEAD | 2.0 CM OR MORE AND LESS | 4.0 CM OR MORE |
| HEAD REGIONS | POSTURE | THAN 4.0 CM | |
| | TRANSVERSE | 2.5 CM OR MORE AND LESS | 3.0 CM OR MORE |
| | OVERSHIFT OF | THAN 3.0 CM | |
| | HEAD POSITION | | |

When the walking motions are evaluated according to the first criterion presented in TABLE 1 and there is one or more item corresponding to “warning” or “need caution”, for example, it is determined as having the compensatory movement. Based on the extent of “need caution” and “warning” (the extent deviated from a normal range), the number of items corresponding to “need caution” or “warning” in each body region and the like, at least one or more body region as a source for causing the compensatory movement can be inferred.

The flowchart of FIG. 2 will be explained again. When the measurement data does not satisfy the first criterion (No in step S5), a coaching program for eliminating the compensatory movement in the specific movement or the like is displayed on the display screen or the like for having the exerciser try to correct the specific movement or the like by coaching (step S6). Then, an instruction is given to the exerciser to redo the specific movement or the like that is instructed in step S1 (step S7). Then, the specific movement or the like done by the exerciser is monitored again by the measurement device (step S8), and the measurement data regarding the specific movement or the like acquired by the monitoring is transmitted to the computer apparatus 1 from the measurement device (step S9). Note that coaching in step S6 may be given during execution of the specific movement, and step S7 is omitted in such case.

The computer apparatus 1 receives the measurement data regarding the specific movement or the like from the measurement device (step S10), and determines whether or not the measurement data satisfies the first criterion (step S11). When the measurement data satisfies the first criterion (Yes in step S11), a function improving exercise program for maintaining the compensatory movement eliminated state is displayed on the display screen (step S12), for example, and then, the processing is terminated. Note that the content of the function improving program in step S12 may be the same as the coaching program presented in step S6.

When the measurement data does not satisfy the first criterion (No in step S11), the computer apparatus 1 gives an instruction to the exerciser to do the basic movement (step S13). The types of basic movements to be done may be determined based on the degree of the measurement data regarding the specific movement or the like unsatisfying the first criterion or may be determined based on the compensatory movement pattern. For example, through the exercise using a joint in the periphery of the region where the compensatory movement pattern is observed, for example, the source body region of the compensatory movement can be identified more accurately. Then, the basic movement done by the exerciser is monitored by the measurement device (step S14), and the measurement data regarding the basic movement acquired by the monitoring is transmitted to the computer apparatus 1 from the measurement device (step S15).

The computer apparatus 1 receives the measurement data regarding the basic movement or the like from the measurement device (step S16), and determines whether or not the measurement data satisfies the second criterion (step S17). When the measurement data satisfies the second criterion (Yes in step S17), the processing is returned to step 1 again to recheck whether or not there is the compensatory movement observed in the specific movement or the like.

Now, the second criterion will be described. With the second criterion, existence of the compensatory movement in the periarticular used for conducting the basic movement is determined. The joints to be used vary depending on the types of the basic movement, so that it is preferable, when monitoring the basic movement, to use a measurement device capable of monitoring motions and the like of the joints and the peripheral region thereof used for conducting the basic movement. For example, when using a motion capture as the measurement device, it is possible to employ the attaching positions in monitoring of the specific movement or the like described above as the attaching positions of the sensors or the like in a necessary base.

When overhead squat is to be conducted as the basic movement, existence of the compensatory movement is determined in following four points of view. First point of view is whether or not motions of the feet and foot joints are normal. The second point of view is whether or not motions of the knees are normal. The third point of view is whether or not motions of lumbo-pelvic-hip complex are normal. The fourth point of view is whether or not motions of the shoulders, head and cervical vertebra are normal.

While examples of the second criterion in a case where the basic movement is overhead squat are presented in TABLE 2, the second criterion is not limited to those presented in TABLE 2.

TABLE 2

| | COMPENSATORY | | |
|---|---|---|---|
| BODY REGION | MOVEMENT PATTERN | DETERMINATION POINT | NORMAL |
| FOOT REGION AND | TOE FACING | ANGLE BETWEEN TRAVEL DIRECTION AND LINE | 0° OR MORE AND |
| FOOT JOINT | OUTWARD | PASSING THROUGH INTERMEDIATE POINT OF | LESS THAN 13° |
| | | SECOND TOE AND THIRD TOE AND HEEL | |
| | TOE FACING | ANGLE BETWEEN TRAVEL DIRECTION AND LINE | 0° OR MORE AND |
| | INWARD | CONNECTING INTERMEDIATE POINT OF SECOND | LESS THAN 13° |
| | | TOE AND THIRD TOE AND HEEL | |
| | ARCH CRUSHED | ANGLE BETWEEN MAJOR AXIS OF TIBIA AND | 5° OR MORE AND |
| | | CALCANEUS BONE | LESS THAN 14° |
| | SUPINATION OF | ANGLE BETWEEN MAJOR AXIS OF TIBIA AND | 5° OR MORE AND |
| | FOOT REGION | CALCANEUS BONE | LESS THAN 14° |
| | ELEVATION | PERPENDICULAR DISTANCE BETWEEN FLOOR | 0 CM |
| | OF HEEL | PLANE AND HEEL SOLE | |
| KNEE JOINT | KNEE IN | ANGLE BETWEEN MAJOR AXIS OF FEMUR IN | 170° OR MORE AND |
| | | FRONTAL PLANE AND MAJOR AXIS OF TIBIA | LESS THAN 180° |
| | KNEE OUT | ANGLE BETWEEN MAJOR AXIS OF FEMUR IN | 170° OR MORE AND |
| | | FRONTAL PLANE ANO MAJOR AXIS OF TIBIA | LESS THAN 180° |
| PELVIS AND | OVER ANTERIOR TILT, | ANGLE BETWEEN HORIZONTAL PLANE PASSING | 32° OR MORE AND |
| LUMBAR REGION | OVEREXTENSION | THROUGH ANTERIOR SUPERIOR ILIAC SPINE AND LINE | LESS THAN 37° |
| | OF LUMBER VERTEBRA | PASSING THROUGH ANTERIOR SUPERIOR ILIAC | |
| | | SPINE AND POSTERIOR SUPERIOR ILIAC SPINE | |
| | OVER POSTERIOR | ANGLE BETWEEN HORIZONTAL PLANE PASSING | 32° OR MORE AND |
| | TILT, FLEXION | THROUGH ANTERIOR SUPERIOR ILIAC SPINE AND LINE | LESS THAN 37° |
| | | PASSING THROUGH ANTERIOR SUPERIOR ILIAC | |
| | | SPINE AND POSTERIOR SUPERIOR ILIAC SPINE | |
| | UPPER BODY BEING | ANGLE BETWEEN PERPENDICULAR LINE PASSING | 42° OR MORE AND |
| | BENT FORWARD | THROUGH GREATER TROCHANTER AND LINE | LESS THAN 48° |
| | | PASSING FROM PARIETAL REGION OF HEAD | |
| | | THROUGH FEMORAL REGION | |
| | UPPER BODY BECOMING | ANGLE BETWEEN PERPENDICULAR LINE PASSING | 42° OR MORE AND |
| | CLOSE TO PERPENDICULAR | THROUGH GREATER TROCHANTER AND LINE | LESS THAN 48° |
| | | PASSING FROM PARIETAL REGION OF HEAD | |
| | | THROUGH FEMORAL REGION | |
| | TRANSVERSE OVERTILT | ANGLE BETWEEN HORIZONTAL PLANE AND LINE | LESS THAN 3° |
| | OF PELVIS (TRANSVERSE | PASSING THROUGH LEFT POSTERIOR SUPERIOR | |
| | WEIGHT SHIFT) | ILIAC SPINE ANO RIGHT POSTERIOR SUPERIOR | |
| | | ILIAC SPINE | |
| SHOULDER, HEAD | ARM DROPPING DOWN | LINE CONNECTING ACROMION AND GREATER | −5° OR MORE |
| REGIONS, AND | FORWARD | TROCHANTER OF FEMUR AND LINE CONNECTING | AND LESS THAN |
| CERVICAL VERTEBRA | | ACROMION AND ULNAR STYLOID PROCESS WHEN | 10° |
| | | VIEWED FROM SIDE | |

| | COMPENSATORY | | |
|---|---|---|---|
| BODY REGION | MOVEMENT PATTERN | WARNING | NEED CAUTION |
| FOOT REGION AND | TOE FACING | 13° OR MORE AND | 20° OR MORE |
| FOOT JOINT | OUTWARD | LESS THAN 20° | |
| | TOE FACING | −8° OR MORE AND | LESS THAN −8° |
| | INWARD | LESS THAN 0° | |
| | ARCH CRUSHED | 14° OR MORE AND | 28° OR MORE |
| | | LESS THAN 20° | |
| | SUPINATION OF | −3° OR MORE AND | LESS THAN −3° |
| | FOOT REGION | LESS THAN 5° | |
| | ELEVATION | 0 CM OR MORE | 2.5 CM OR MORE |
| | OF HEEL | AND LESS THAN 2.5 CM | |
| KNEE JOINT | KNEE IN | 160° OR MORE AND | LESS THAN 160° |
| | | LESS THAN 170° | |
| | KNEE OUT | 180° OR MORE AND | 190° OR MORE |
| | | LESS THAN 190° | |
| PELVIS AND | OVER ANTERIOR TILT, | 37° OR MORE AND | 41° OR MORE |
| LUMBAR REGION | OVEREXTENSION | LESS THAN 41° | |
| | OF LUMBER VERTEBRA | | |
| | OVER POSTERIOR | 27° OR MORE AND | LESS THAN 27° |
| | TILT, FLEXION | LESS THAN 32° | |
| | UPPER BODY BEING | 48° OR MORE AND | 54° OR MORE |
| | BENT FORWARD | LESS THAN 54° | |
| | UPPER BODY BECOMING | 36° OR MORE AND | LESS THAN 36° |
| | CLOSE TO PERPENDICULAR | LESS THAN 42° | |
| | TRANSVERSE OVERTILT | 3° OR MORE AND | 7° OR MORE |
| | OF PELVIS (TRANSVERSE | LESS THAN 7° | |
| | WEIGHT SHIFT) | | |
| SHOULDER, HEAD | ARM DROPPING DOWN | 10° OR MORE AND | 30° OR MORE |
| REGIONS, AND | FORWARD | LESS THAN 30° | |
| CERVICAL VERTEBRA | | | |

When the overhead squat is evaluated according to the second criterion presented in TABLE 2 and there is one or more item corresponding to “warning” or “need caution”, for example, it is determined as having a compensatory movement. Based on the extent of “need caution” and “warning” (the extent deviated from a normal range), the number of items corresponding to “need caution” or “warning” in each body region and the like, at least one or more body region as a source for causing the compensatory movement can be inferred.

Other than the compensatory movement patterns presented in TABLE 2, there are also compensatory movement patterns such as shift in the center-of-mass asymmetrical to the lumbo-pelvic-hip (buttocks shift either to left or right), protrusion of abdomen, bending of elbow, upward rotation of scapula, protraction of scapula (region between the scapulae becomes broadened), elevation of shoulder, and winged scapula and forward head position, for example, so that it is preferable to conduct monitoring to be able to detect those compensatory movement patterns.

For each of the compensatory movement patterns detected by overhead squat, examples of muscles that may have tonus and weakening as well as the function improving exercise programs are presented in TABLE 3. As the function improving exercise programs, other than those written in TABLE 3, it is preferable to include also means for lightening hypertonic of the muscles, such as giving massages, stretching, and the like for the muscles that may have muscle tonus.

TABLE 3

| | | | EXAMPLES OF |
|---|---|---|---|
| COMPENSATORY | | | FUNCTION |
| MOVEMENT | | MUSCLE WEAKENING | IMPROVING EXERCISE |
| PATTERN | MUSCLE TONE (TIGHTNESS) | (WEAKNESS) | PROGRAM |
| ARCH | LATERAL HEAD OF GASTROCNENIUS | GLUTEUS MEDIAS MUSCLE, | TUBE WALKING, |
| CRUSHED | MUSCLE, FIBULARIS MUSCLE | ANTERIOR TIBIAS MUSCLE, | BALL-BRIDGE, |
| | | POSTERIOR TIBIAL MUSCLE | SINGLE-LEG-BALANCE |
| | | | REACH, SINGLE-LEG |
| | | | SQUAT |
| TOE FACING | SOLEUS MUSCLE, BICEPS FEMORIS | GLUTEUS MEDIUS MUSCLE | TUBE WALKING, |
| OUTWARD | MUSCLE, PIRIFORMIS MUSCLE | | BALL-BRIDGE, |
| | | | SINGLE-LEG-BALANCE |
| | | | REACH, SINGLE-LEG |
| | | | SQUAT |
| KNEE IN | ADDUCTORS, ILIOTIBIAL BAND | GLUTEUS MEDIUS MUSCLE, | TUBE WALKING, |
| | | GLUTEUS MAXIMUS MUSCLE | BALL-BRIDGE, |
| | | | SINGLE-LEG-BALANCE |
| | | | REACH, SINGLE-LEG |
| | | | SQUAT |
| KNEE OUT | BICEPS FEMORIS MUSCLE, ILIOPSOAS | GLUTEUS MEDIUS MUSCLE, | TUBE WALKING, |
| | MUSCLE, PIRIFORMIS MUSCLE | GLUTEUS MAXIMUS MUSCLE | BALL-BRIDGE, |
| | | | SINGLE-LEG-BALANCE |
| | | | REACH, SINGLE-LEG |
| | | | SQUAT |
| WEIGHT SHIFT | BICEPS FEMORIS MUSCLE(RIGHT), | GLUTEUS MAXIMUS MUSCLE | TUBE WALKING, |
| ASYMMETRICAL | ILIOPSOAS MUSCLE (RIGHT), | (RIGHT), GLUTEUS MEDIUS | BALL-BRIDGE, |
| TO LUMBER | PIRIFORMIS MUSCLE (RIGHT), | MUSCLE(LEFT), TRANSVERSE | SINGLE-LEG-BALANCE |
| VERTEBRA, | EXTERNAL OBLIQUE MUSCLE | ABDOMINAL MUSCLE, | REACH, SINGLE-LEG |
| PELVIS AND | (RIGHT), FIBULARIS MUSCLE (LEFT), | MULTIFIOUS MUSCLE | SQUAT |
| COXA (CASE | SOLEUS MUSCLE (LEFT), | | |
| WHERE | ADDUCTORS (LEFT), ILIOTIBIAL | | |
| BUTTOCKS | BAND (LEFT), INTERNAL | | |
| SHIFT TO | OBLIQUE MUSCLE (LEFT) | | |
| RIGHT) | | | |
| OVER ANTERIOR | ILIOPSOAS MUSCLE, RECTUS FEMORIS | GLUTEUS MAXIMUS MUSCLE, | TUBE WALKING, |
| TILT, SWAY BACK | MUSCLE, ERECTOR SPINAE MUSCLE, | GLUTEUS MEDIUS MUSCLE, | BALL-BRIDGE, |
| (INCREASE IN | LATISSIMUS DORSI MUSCLE | PELVIS STABILIZATION | BALL-CRUNCH, SINGLE- |
| EXTENSION OF | | MUSCLE GROUP | LEG-BALANCE REACH, |
| LUMBER | | | SINGLE-LEG SQUAT |
| VERTEBRA) | | | |
| OVER POSTERIOR | EXTERNAL OBLIQUE MUSCLE, RECTUS | GLUTEUS MAXIMUS MUSCLE, | TUBE WALKING, |
| TILT, FLEXION | ABDOMINIS MUSCLE, BICEPS FEMORIS | GLUTEUS MEDIUS MUSCLE, | BAIL-BRIDGE, |
| (INCREASE | MUSCLE, SEMIMEMBRANEOUS | PELVIS STABILIZATION | BALL-CRUNCH, |
| IN FLEXION OF | MUSCLE, SEMITENDINOSUS MUSCLE | MUSCLE GROUP | SINGLE-LEG- BALANCE |
| LUMBER | | | REACH, SINGLE- |
| VERTEBRA) | | | LEG SQUAT |
| PROTRUSION | ILIOPSOAS MUSCLE | PELVIS STABILIZATION | TUBE WALKING, |
| OF ABDOMEN | | MUSCLE GROUP | BALL-BRIDGE, |
| | | | BALL-COBRA, SINGLE- |
| | | | LEG-BALANCE REACH, |
| | | | SINGLE-LEG SQUAT |
| OVEREXTENSION | LATISSIMUS DORSI MUSCLE, | MEDIAL REGION OF TRAPEZIUS, | BALL-COBRA, |
| OF LUMBER | PECTORALIS MAJOR MUSCLE | INFERIOR REGION OF TRAPEZIUS | BALL-SCAPTION, |
| VERTEBRA | | | BALL PNF, SINGLE- |
| OR ARM | | | LEG WINDMILL |
| DROPPING | | | |
| FORWARD | | | |
| ELBOW | LATISSIMUS DORSI MUSCLE, | MEDIAL REGION OF TRAPEZIUS, | BALL-COBRA, |
| BEING BENT | PECTORALIS MAJOR MUSCLE | INFERIOR REGION OF TRAPEZIUS | BALL-SCAPTION, |
| | | | BALL PNF, SINGLE- |
| | | | LEG WINDMILL |
| SUPERIOR | SUPERIOR REGION OF TRAPEZIUS, | RHOMBOID MUSCLE, | BALL-COBRA, |
| ROTATION OF | LEVATOR MUSCLE OF SCAPULA, | MEDIUS REGION OF TRAPEZIUS, | BALL-SCAPTION, |
| SCAPULA | PECTORALIS MAJOR MUSCLE, | INFERIOR REGION OF TRAPEZIUS | BALL PNF, SINGLE- |
| | PECTORALIS MINOR MUSCLE | | LEG WINDMILL |
| ABDUCTION | PECTORALIS MAJOR MUSCLE, | RHOMBOID MUSCLE, | BALL-COBRA, |
| OF SCAPULA | PECTORALIS MINOR MUSCLE, | MEDIUS REGION OF TRAPEZIUS, | BALL-SCAPTION, |
| | LATISSIMUS DORSI MUSCLE | INFERIOR REGION OF TRAPEZIUS, | BALL PNF, SINGLE- |
| | | TERES MINOR MUSCLE, | LEG WINDMILL |
| | | INFRASPINATUS MUSCLE | |
| ELEVATION OF | SUPERIOR REGION OF TRAPEZIUS, | INFERIOR REGION OF TRAPEZIUS | BALL-COBRA, |
| SHOULDER | LEVATOR MUSCLE OF SCAPULA | | BALL-SCAPTION, |
| | | | BALL PNF, SINGLE- |
| | | | LEG WINDMILL |
| WINGED | PECTORALIS MINOR MUSCLE | SERRATUS ANTERIOR MUSCLE, | BALL-COBRA, |
| SCAPULA | | INFERIOR REGION OF TRAPEZIUS | BALL-SCAPTION, |
| | | | BALL PNF, SINGLE- |
| | | | LEG WINDMILL |
| HEAD SHIFTED | STERNOCLEIDOMASTOID MUSCLE, | DEEP REGION OF CERVICAL, | BALL-CERVICAL |
| FORWARD | SCALENE MUSCLE | FLEXOR (LONGUS COLLI | VERTEBRA |
| | | MUSCLE, LONGUS CAPITIS) | RETRACTION |

The flowchart of FIG. 2 will be explained again. When the measurement data does not satisfy the second criterion (No in step S17), a coaching program for eliminating the compensatory movement in the basic movement is displayed on the display screen or the like for having the exerciser try to correct the basic movement by coaching (step S18). Then, an instruction is given to the exerciser to redo the basic movement that is instructed in step S13 (step S19). Then, the basic movement done by the exerciser is monitored again by the measurement device (step S20), and the measurement data regarding the basic movement and acquired by the monitoring is transmitted to the computer apparatus 1 from the measurement device (step S21). Note that coaching in step S18 may be given during execution of the basic movement, and step S19 is omitted in such case.

The computer apparatus 1 receives the measurement data regarding the basic movement from the measurement device (step S22), and determines whether or not the measurement data satisfies the second criterion (step S23). When the measurement data satisfies the second criterion (Yes in step S23), a function improving exercise program for maintaining the compensatory movement eliminated state is displayed on the display screen (step S24), for example, and then, the processing is terminated. Note that the content of the function improving program in step S24 may be the same as the coaching program presented in step S18.

When the measurement data does not satisfy the second criterion (No in step S23), the types of separate movement or the like to be done by the exerciser may be determined based on the degree of the measurement data regarding the basic movement unsatisfying the second criterion or may be determined based on the compensatory movement pattern and the like (step S25). For example, when the compensatory movement is observed in the foot region in step S23, selected are the separate movement or the like for evaluating the mobility and stability of the foot region. Note that the types of the separate movements and the like and the priority order may be determined based on the degree of the measurement data regarding the specific movement or the like unsatisfying the first criterion, may be determined based on the compensatory movement pattern, or may be determined based on both of the measurement data regarding the specific movement or the like and the measurement data regarding the basic movement.

Before executing step S25, it is preferable to determine the function improving exercise program directed to eliminate the compensatory movement by inferring the source body region and the characteristic of the compensatory movement according to the measurement data regarding the basic movement, for example, and display the function improving exercise program on the display screen. When there is improvement observed in the compensatory movement in the basic movement through having the exerciser execute the function improving exercise program, it is desirable to conduct monitoring of the basic movement again after continuing the function improving exercise program for about three to twelve weeks, for example. Meanwhile, when no improvement is observed, it is preferable to execute an individual exercise program after conducting an evaluation of the separate movement or the like and identifying the source body region.

Then, an instruction is given to the exerciser via a determination display screen or the like to do the separate movement or the like determined in step S25 (step S26). Then, the separate movement or the like done by the exerciser are monitored by the measurement device (step S27), and the measurement data regarding the separate movement or the like acquired by the monitoring is transmitted to the computer apparatus 1 from the measurement device (step S28).

The computer apparatus 1 receives the measurement data regarding the separate movement or the like from the measurement device (step S29), and identifies the body region to be the source for causing the compensatory movement based on whether or not the measurement data satisfies the third criterion (step S30). Then, a function improving exercise program for eliminating the compensatory movement is displayed on the display screen according to the body region to be the source for causing the compensatory movement (step S31), for example, and then, the processing is terminated.

About a group of steps regarding the specific movement or the like presented as step S1 to step S12 and a group of steps regarding the basic movement presented as step S13 to step S24 of the flowchart of FIG. 2 described above, only one of the group of steps may be executed, and such mode is also included in the embodiment of the present invention.

Now, the separate movements, static postures, and the third criterion will be described. The separate movements are broadly classified into movements for evaluation of the lower limbs, movements for evaluation of the trunk, and movements for evaluation of the upper limbs.

Examples of the movements for evaluation of the lower limbs may be flexion and expansion of the metatarsophalangeal joint of great toe (evaluation of foot region), planter flexion and dorsal flexion of the talocrural joint (evaluation of foot joint), flexion and expansion of the knee (evaluation of the knee joint), medial rotation and lateral rotation of the coxa (evaluation of the coxa), torsion of the femur (evaluation of the coxa), Thomas test (evaluation of the coxa), standing on all fours with lock back state (evaluation of the coxa and the pelvis), and holding both arms in a dorsal position and lifting up and moving both feet or one of the feet away from the floor surface.

With flexion and expansion of the knee, the range of motion of the knee joint is measured. Flexion and expansion of the knee may be done and measured in a dorsal position or in a prone position. There is no specific limit set for the use of the measurement device; however, when using a goniometer, its axis (center) of the goniometer is set to the femur lateral condyle, a fixed arm is set to the center line of the femur passing through the greater trochanter, and a movable arm is set to the center line of the fibula passing through the lateral malleolus to measure the range of motion of the knee joint.

With the movement of holding both arms in a dorsal position and lifting up and moving both feet or one of the feet away from the floor surface, used as the criterion are whether or not there is anterior pelvic tilt or sway back, or elevation of the inferior region of the ribs, and the like.

Examples of the movement for evaluation of the trunk may be repetition of breathing in a dorsal position, axial rotation by taking a dorsal position on an exercise support tool, elevation of arms and legs by taking a dorsal position or a prone position on an exercise support tool, and lifting up, from the floor, an arm and a leg on a diagonal line in a posture on all fours. As the exercise support tool, it is preferable to use a substantially cylindrical or substantially semicylindrical shaped tool. Examples may be a StretchPole® and a half-cut StretchPole® that are products of LPN Corporation.

With repetition of breathing in a dorsal position, used as the criterion are whether or not the abdominal region and the like are used as a whole when breathing in, whether or not there is natural contraction of the abdominal region when breathing in, whether or not there is elevation of the shoulder, whether or not there is a move in the pelvis, whether or not there is broadwise spreading of the inferior region of the ribs, and the like.

With the movement of elevating the legs and arms by taking a dorsal position on the exercise support tool, used as the criterion are whether or not there is distortion in the head, the thorax and the pelvis, whether or not there is no unnecessary strain and being stable, and the like. Note that similar criterion can be used for elevation of arms and legs done by taking a prone position on the exercise support tool.

With the movement of standing on all fours and lifting up the arm and the leg on a diagonal line from the floor, used as the criterion are whether or not elevation of the leg and arm can be done while keeping the pelvis and the lumber region stable, whether or not there is rotation of the pelvis and the thorax, whether or not there is winging of scapula, and the like.

Examples of the movement for evaluation of the upper limbs may be shoulder flexion (evaluation of the shoulder and the scapula), shoulder abduction (evaluation of the shoulder and the scapula), shoulder rotation (evaluation of the shoulder and the scapula), cervical vertebra flexion (evaluation of the neck region), cervical vertebra expansion (evaluation of the neck region), cervical vertebra lateral bending and cervical vertebra rotation (evaluation of the neck region), and a movement of elevating and moving both arms while lifting up both legs from the floor in a dorsal position.

With the movement of elevating and moving both arms while lifting up both legs from the floor in a dorsal position, used as the criterion are whether or not there is anterior pelvic tilt or sway back, whether or not the upper limb can be bent at 170 degrees or more without elevation of the inferior region of the ribs, and the like.

For the criterion regarding the movement for evaluation of the lower limbs, the movement for evaluation of the trunk, and the movement for evaluation of the upper limbs, it is also possible to employ the conventionally used evaluation criterion other than the criterion described above. Further, there may be a plurality of movements to be done, and what type of separate movement is to be done may be determined based on the degree of the measurement data regarding the basic movement unsatisfying the second criterion, based on the compensatory movement pattern, or the like. Furthermore, without limiting to the movements described above, movements of the regions the coach or the trainer thinks necessary may be added as well.

Evaluation of a static posture may be conducted by having the exerciser take a posture of standing, sitting, or kneeling position, for example, and monitoring the position of a prescribed body region of the exerciser in a still state of the posture. With the evaluation of the static posture, it is possible to acquire the information of the region that can be the source body region of the compensatory movement. Thus, when candidates for the source body region of the compensatory movement cannot be identified from the measurement data of the separate movement or the like and/or the basic movement, for example, cause factors of the compensatory movement can be extracted through conducting evaluation of the static posture. Further, even when candidates for the source body region of the compensatory movement can be identified from the measurement data of the separate movement or the like and/or the basic movement, evaluation of the static posture may be conducted before measuring the separate movement in terms of improving the accuracy for identifying the final source body region or providing the function improving exercise program suited for the exerciser through capturing the characteristic and nature of the compensatory movement. After extracting the cause factors of the compensatory movement by the evaluation of the static posture, it is preferable to conduct monitoring of the separate movement as well in order to grasp the state of the source body region accurately and to provide the more appropriate function improving program.

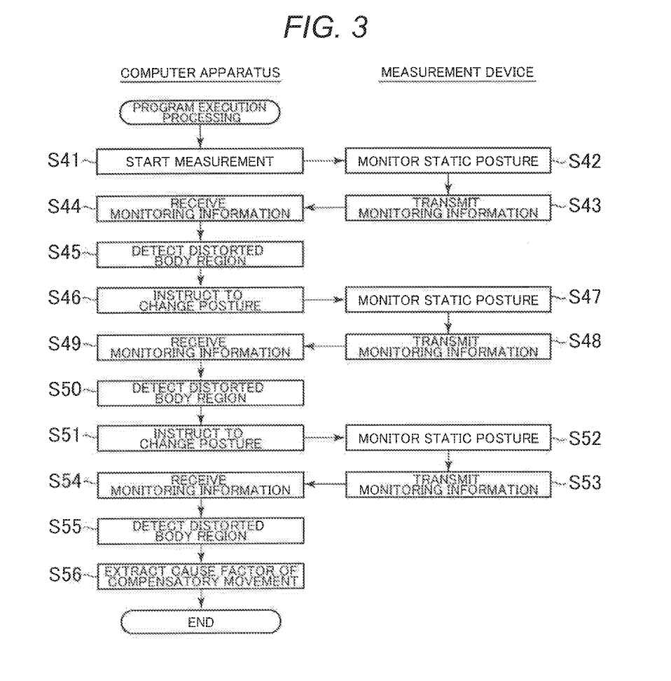

Now, program execution processing for extracting the cause factors of the compensatory movement from the evaluation of the static posture corresponding to at least one of the first embodiment will be described. FIG. 3 is a flowchart of the program execution processing corresponding to at least one of the first embodiment of the present invention.

While a case of having the exerciser take a standing position as the static posture and observing the body of the exerciser from a front side will be described hereinafter as a way of example, the static posture is not limited to the standing position and also the body of the exerciser may be observed from a lateral side or a back side.

First, upon accepting an operation input or the like of an exerciser or a trainer, the computer apparatus 1 starts the measurement processing (step S41). Then, the exerciser is encouraged to take a standing position for monitoring the position of a prescribed body region of the exerciser (step S42), and the measurement data regarding the standing position acquired by the monitoring is transmitted to the computer apparatus 1 from the measurement device (step S43).

The computer apparatus 1 receives the measurement data regarding the standing position from the measurement device (step S43), and evaluate the measurement data based on the second criterion to detect a distorted body region of the exerciser (step S45).

Now, steps S42 to S45 will be described in more detail. Evaluation of the static posture can be conducted through having the exerciser stand upright at a prescribed position with the second toes of both feet facing toward front side while being parallel to each other and directly view a designated object at a height of eye level, connecting positions of prescribed body regions of the exerciser to make four blocks that are a head block, a thorax block, a pelvis block, and a foot block, and evaluating states or positional relationships of the four blocks.

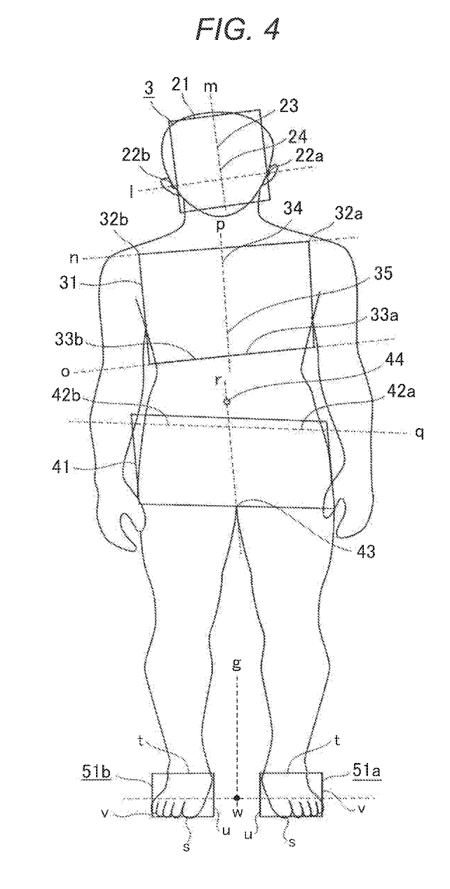

FIG. 4 is a schematic view illustrating an example of a block forming method when an exerciser 3 is in a standing position. A head block 21 is formed by taking a line 1 connecting external acoustic openings 22a and 22b on left and right as a transverse reference line, taking a face median line m connecting a glabella 23 and a philtrum 24 as a vertical reference line, and translating each of the reference lines to fit the outer ring of the skull. A thorax block 31 takes each of a line n connecting acromia 32a and 32b on left and right and a line o connecting costal arches 33a and 33b on left and right as transverse line. Then, a line p connecting a manubrium sterni jugular notch 34 and an ensiform process 35 is taken as a vertical reference line, and the vertical reference line is translated along the outer ring of the ribs.

A pelvis block 41 is formed by taking a line connecting iliac crests 42a and 42b on left and right as a transverse reference line q, taking a line connecting a pubic symphysis 43 and a navel 44 as a vertical reference line r, and translating each of the reference lines to fit the outer frame of the pelvis. Left and right foot blocks 51a and 51b can be formed by looking at the whole outer frame of the foot region and, for example, can be formed with a horizontal line s passing through the tips of toes, a horizontal line t passing through medial malleoli, a perpendicular line u passing through the medial malleoli, and a perpendicular line v passing through the lateral margins of the metatarsophalangeal joints. Further, a line passing through a medial point w of a segment connecting each of the center points of the foot blocks 51a and 51b and vertical to the floor surface is taken as a center-of-mass point g. Although not illustrated in the drawing, it is also possible to form a transverse line connecting knee joints (centers of patellae) on left and right in addition to the four blocks described above to be used for evaluation of the static posture.

When conducting monitoring by attaching the sensor or the like, it is preferable to attach the sensor at the points (e.g., external acoustic openings 22a and 22b, the acromia 32a and 32b) as the origins for making the vertical and transverse lines or the reference lines described above.

Evaluation of each of the blocks is conducted based on whether or not the transverse line forming each of the blocks is parallel to the floor surface (whether or not the block is tilted either to left or right), distance between the reference point corresponding to each of the blocks and the center-of-mass line, and the like. A case where the transverse line is not parallel to the floor surface is considered as abnormal, and the block (body region corresponding to the block) is determined to have distortion. The transverse line connecting the knee joints (centers of patellae) on left and right is also considered abnormal when not parallel to the floor surface, and it is determined that there is distortion in the knee region in such case.

Further, regarding the distance between the reference point corresponding to each of the blocks and the center-of-mass line, a case of 7 mm or more is considered as abnormal, for example, and it is decided that the block has distortion. For example, a case of 7 mm or more and less than 12 mm is defined as abnormal level 1, a case of 12 mm or more and less than 17 mm is defined as abnormal level 2, and a case of 17 mm or more and less than 22 mm is defined as abnormal level 3 to determine the abnormal level, and the most distorted block can be identified according to the abnormal levels. Examples of “reference points” may be the glabella in case of the head block, may be the manubrium sterni jugular notch or the ensiform process in case of the thorax block, may be the pubic symphysis in case of the pelvis block, may be the intermediate point of the knee joints on left and right in case of the knee region, and the medial point of the taluses on left and right in case of the foot regions.

In FIG. 4, it is observed that the head block 21 is tilted to right, the thorax block 31 is tilted to right, and the pelvis block 41 is tilted to left.

Even when tilt or the like is seen in a certain block, it does not lead to identifying the distorted body region in a posture that may be a cause of the compensatory movement; therefore, the body regions to be the cause for inducing distortion of the upright posture in step S45 are narrowed down in step S46 and thereafter.

The flowchart of FIG. 3 will be explained again. The computer apparatus 1 gives an instruction to the exerciser to change the posture via the display screen or the like according to the distorted body region detected in step S45 (step S46). As the change of the posture, for example, it is preferable to change to a sitting position so that the pelvis comes neutral in order to identify whether the body region as a cause of the compensatory movement is in the upper body or the lower body. Hereinafter, a case of changing the posture to a sitting position will be described.

The exerciser is encouraged to take a sitting position to monitor positions of prescribed body regions of the exerciser (step S47), and the measurement data regarding the sitting position acquired by the monitoring is transmitted to the computer apparatus 1 from the measurement device (step S48). The computer apparatus 1 receives the measurement data regarding the sitting position from the measurement device (step S49), and evaluates the states of each of the blocks and the positional relationships by a similar method as step S45 to detect the distorted body region of the exerciser (step S50). Then, in order to narrow down the body regions as the cause for inducing distortion of the upright posture in step S45, an instruction is given to the exerciser to change the posture via the display screen or the like depending on the detected distorted body region or depending on whether or not the distortion is disappeared (step S51). Examples of the change of the posture may include changing to a kneeling position, closing eyes while keeping the sitting position, giving a voice guidance such as “slightly pull down the right shoulder”, and making the thorax neutral so that the thorax block and the pelvis block face the same direction through a guidance or the like given by a coach or a trainer.

The exerciser is encouraged to change the posture to monitor positions of prescribed body regions of the exerciser (step S52), and the measurement data regarding the changed posture acquired by the monitoring is transmitted to the computer apparatus 1 from the measurement device (step S53). The computer apparatus 1 receives the measurement data regarding the changed posture from the measurement device (step S54), and evaluates the states of each of the blocks and the positional relationships by a similar method as step S45 to detect the distorted body region of the exerciser (step S55). Then, a cause factor of the compensatory movement is extracted according to the detected distorted body region or according to whether or not the distortion is disappeared (step S56), and the processing is terminated.

Now, steps S50 to S56 will be described by referring to a specific example. For example, when it is found with the measurement in a standing position that there is distortion in the thorax and also found with the measurement in a sitting position that there is distortion in the thorax, the source body region of the compensatory movement is in the cervix region or the lumber/abdomen region. In such case, an instruction for changing the posture is given to close eyes while keeping the sitting position, a voice guidance such as “slightly pull down the right shoulder” or the like is given, and an instruction is given to make the thorax neutral so that the thorax block and the pelvis block face the same direction through a guidance or the like given by the coach or the trainer. When it is found that distortion of all the blocks is disappeared as a result of the measurement in such posture, the lumbar region and the abdomen region are extracted as the cause factors of the compensatory movement. Further, when distortion in the head is detected as a result of the measurement in such posture, the cervix region is extracted as the cause factor of the compensatory movement.

Further, when there is distortion found in the thorax with the measurement in a standing position and there is no distortion found in the thorax with the measurement in a sitting position, the foot region or the coxa region is extracted as the cause factor of the compensatory movement. In such case, an instruction is given to change the posture to a kneeling position, for example. When distortion in the thorax is not detected as a result of the measurement in such posture, the foot region is extracted as the cause factor of the compensatory movement. Further, when distortion in the thorax is detected as a result of the measurement of such posture, the coxa region is extracted as the cause factor of the compensatory movement.

### OTHER EMBODIMENTS

In the embodiments above, mainly described is the case where the computer apparatus connected to the measurement device executes various types of steps such as the step of determining existence of the compensatory movement in the first motion, the step of determining existence of the compensatory movement in the second motion, the step of determining the type of the third motion, and the step of identifying the body region to be the source for causing the compensatory movement; however, those steps may be executed not by the computer apparatus connected to the measurement device but by a server device connected to the computer apparatus via communication. In such case, it is possible to store the measurement data regarding the still state or moving state of the exerciser and the body regions and the like to be the source for causing the identified compensatory movement in the server device while being associated with the exerciser. Through storing the measurement data and the body regions and the like to be the source for causing the identified compensatory movement, it is possible to check chronological passage of suppression of the compensatory movement of the exerciser by executing the function improving exercise program for eliminating the compensatory movement.

While the embodiment above is configured to measure the still state or the moving state of the exerciser based on the measurement device, the still state or the moving state of the exerciser may be measured by visual inspection of the trainer without using the measurement device, for example. Further, various types of steps such as the step of determining existence of the compensatory movement in the first motion, the step of determining existence of the compensatory movement in the second motion, the step of determining the type of the third motion, and the step of identifying the body region to be the source for causing the compensatory movement can be executed by the trainer without using the computer apparatus and the server device.

That is, the embodiment includes the compensatory movement source body region identification method that includes a step of first determination that determines existence of the compensatory movement in the first motion based on whether or not the still state or the moving state of a first measurement body region group of the exerciser in the first motion satisfies the first criterion while having the exerciser conduct the first motion, and/or a step of second determination that determines existence of the compensatory movement in the second motion that is of lower complicity than the first motion based on whether or not the still state or the moving state of a second measurement body region group of the exerciser in the second motion satisfies the second criterion; and a step of identifying the body region to be the source for causing the compensatory movement based on whether or not the still state or the moving state of the third measurement body region group of the exerciser satisfies the third criterion while having the exerciser conduct the third motion that is of lower complicity than the second motion, when determined in the step of first determination that determines and/or the step of second determination that determines that there is the compensatory movement. For executing the compensatory movement source body region identification method, it is possible to use a source body region identification system that includes the first recording medium recording the first criterion for determining the existence of the compensatory movement in the first motion for the first measurement body region group of each exerciser, for example, and/or the second recording medium recording the second criterion for determining the existence of the compensatory movement in the second motion for the second measurement body region group of each exerciser, and the third recording medium recording the third criterion for identifying the body region to be the source for causing the compensatory movement based on the still state or the moving state in the second measurement body region group of the exerciser.

As the first recording medium, there is no specific limit set for its form as long as the medium is capable of recording information and the trainer can acquire the information, and examples thereof may be paper, or a terminal with a display device, such as a personal computer or a smartphone. Further, an audio producing device or the like capable of acquiring information by sound or the like may be used as well. Similarly, as the second recording medium and the third recording medium, there is no specific limit set for their forms as long as the media are capable of recording information and the trainer can acquire the information, and examples thereof may be paper, or a terminal with a display device, such as a personal computer or a smartphone, and an audio reproduction device.

In the first recording medium, recorded for each body region are compensatory movement patterns, determination points corresponding to the patterns, and criterion for determining existence of the compensatory movements (e.g., the criterion presented in TABLE 1 or the like), for example. The trainer can determine the existence of the compensatory movement by referring to the information recorded in the first recording medium while having the exerciser conduct the first motion.

In the second recording medium, recorded for each body region are compensatory movement patterns, determination points corresponding to the patterns, and criterion for determining existence of the compensatory movements (e.g., the criterion presented in TABLE 2 or the like), for example. The trainer can determine the existence of the compensatory movement by referring to the information recorded in the second recording medium while having the exerciser conduct the second motion.

In the third recording medium, recorded for each body region to be the source body region of the compensatory movement are criterion for identifying the body region to be the source for causing the compensatory movement based on the still state or the moving state in a third measurement body region group when having the exerciser conduct the third motion. The trainer can identify the body region to be the sauce of the compensatory movement by referring to the information recorded in the third recording medium while having the exerciser conduct the third motion.

### REFERENCE SIGNS LIST

1 COMPUTER APPARATUS
2 COMMUNICATION NETWORK
11 CONTROLLER
12 RAM
13 STORAGE UNIT
14 DISPLAY UNIT
15 INPUT UNIT
16 COMMUNICATION INTERFACE

1. A compensatory movement source body region identification method for identifying a body region to be a source for causing a compensatory movement by using data regarding a still state or a moving state in a prescribed body region of an exerciser measured by one or more measurement device, the method comprising:
first determining that determines existence of the compensatory movement in a first motion based on whether or not first measurement data regarding the still state or the moving state of a first measurement body region group of the exerciser in the first motion satisfies a first criterion; and/or
second determining that determines existence of the compensatory movement in a second motion that is of lower complicity than the first motion based on whether or not second measurement data regarding the still state or the moving state of a second measurement body region group of the exerciser in the second motion satisfies a second criterion; and
identifying at least one or more body region to be the source for causing the compensatory movement based on whether or not third measurement data regarding the still state or the moving state of a third measurement body region group of the exerciser in a third motion that is of lower complicity than the second motion satisfies a third criterion, when determined in the first determining and/or the second determining that there is the compensatory movement.

2. The compensatory movement source body region identification method according to claim 1, wherein the identifying the source body region further identifies a characteristic of the compensatory movement and a proportion contributing to occurrence of the compensatory movement in each of the source body regions.

3. The compensatory movement source body region identification method according to claim 1, further comprising inferring at least one or more body region to be the source for causing the compensatory movement based on whether or not the first measurement data satisfies the first criterion and/or the second measurement data satisfies the second criterion.

4. The compensatory movement source body region identification method according to claim 3, wherein a type of the third motion is decided according to the source inferred to cause the compensatory movement in the inferring the source body region.

5. The compensatory movement source body region identification method according to claim 1, wherein the second determining is executed when determined in the first determining that there is the compensatory movement.

6. The compensatory movement source body region identification method according to claim 1, wherein the measurement device is one kind or more selected from a group consisting of a motion capture, a pressure sensor, an electromyogram meter, an ultrasonic measurement device, and a goniometer.

7. A compensatory movement elimination method for eliminating a compensatory movement, comprising: identifying a body region to be a source for causing the compensatory movement by using the compensatory movement source body region identification method according to claim 1; and having the exerciser do a prescribed exercise depending on the identified body region.

8. A compensatory movement source body region identification system for identifying a body region to be a source for causing a compensatory movement implemented by one or more measurement device measuring data regarding a still state or a moving state in a prescribed body region of an exerciser and a computer apparatus, the system comprising:
a first determinator determining existence of the compensatory movement in a first motion based on whether or not first measurement data regarding the still state or the moving state of a first measurement body region group of the exerciser in the first motion satisfies a first criterion; and/or
a second determinator determining existence of the compensatory movement in a second motion that is of lower complicity than the first motion based on whether or not second measurement data regarding the still state or the moving state of a second measurement body region group of the exerciser in the second motion satisfies a second criterion; and
a source body region identifier identifying at least one or more body region to be the source for causing the compensatory movement based on whether or not third measurement data regarding the still state or the moving state of a third measurement body region group of the exerciser in a third motion that is of lower complicity than the second motion satisfies a third criterion, when determined by the first determiner and/or the second determiner that there is the compensatory movement.

9. The compensatory movement source body region identification system according to claim 8, wherein the source body region identifier further identifies a characteristic of the compensatory movement and a proportion contributing to occurrence of the compensatory movement in each of the source body regions.

10. The compensatory movement source body region identification system according to claim 8, further comprising a source body region inferencer inferring at least one or more body region to be the source for causing the compensatory movement based on whether or not the first measurement data satisfies the first criterion and/or the second measurement data satisfies the second criterion.

11. The compensatory movement source body region identification system according to claim 10, wherein a type of the third motion is decided according to the source inferred to cause the compensatory movement by the source body region inferencer.

12. The compensatory movement source body region identification system according to claim 8, wherein existence of the compensatory movement in the second motion is determined by the second determinator when determined by the first determinator that there is the compensatory movement.

13. The compensatory movement source body region identification system according to claim 8, wherein the measurement device is one kind or more selected from a group consisting of a motion capture, a pressure sensor, an electromyogram meter, an ultrasonic measurement device, and a goniometer.

14. A compensatory movement elimination system eliminating a compensatory movement through identifying a body region to be a source for causing the compensatory movement by using the compensatory movement source body region identification system according to claim 8; and having the exerciser do a prescribed exercise depending on the identified body region.

15. A non-transitory computer-readable recording medium including a program causing a computer apparatus to identify a body region to be a source for causing a compensatory movement by using data regarding a still state or a moving state in a prescribed body region of an exerciser measured by one or more measurement device, the program causing the computer apparatus to function as:
a first determinator determining existence of the compensatory movement in a first motion based on whether or not first measurement data regarding the still state or the moving state of a first measurement body region group of the exerciser in the first motion satisfies a first criterion; and/or
a second determinator determining existence of the compensatory movement in a second motion that is of lower complicity than the first motion based on whether or not second measurement data regarding the still state or the moving state of a second measurement body region group of the exerciser in the second motion satisfies a second criterion; and
a source body region identifier identifying at least one or more body region to be the source for causing the compensatory movement based on whether or not third measurement data regarding the still state or the moving state of a third measurement body region group of the exerciser in a third motion that is of lower complicity than the second motion satisfies a third criterion, when determined by the first determiner and/or the second determiner that there is the compensatory movement.

16. A computer apparatus identifying a body region to be a source for causing a compensatory movement by using data regarding a still state or a moving state in a prescribed body region of an exerciser measured by one or more measurement device, the computer apparatus comprising:
a first determinator determining existence of the compensatory movement in a first motion based on whether or not first measurement data regarding the still state or the moving state of a first measurement body region group of the exerciser in the first motion satisfies a first criterion; and/or
a second determinator determining existence of the compensatory movement in a second motion that is of lower complicity than the first motion based on whether or not second measurement data regarding the still state or the moving state of a second measurement body region group of the exerciser in the second motion satisfies a second criterion; and
a source body region identifier identifying at least one or more body region to be the source for causing the compensatory movement based on whether or not third measurement data regarding the still state or the moving state of a third measurement body region group of the exerciser in a third motion that is of lower complicity than the second motion satisfies a third criterion, when determined by the first determiner and/or the second determiner that there is the compensatory movement.

17. A compensatory movement source body region identification method for identifying a body region to be a source for causing a compensatory movement based on a still state or a moving state in a prescribed body region of an exerciser, the method comprising:
first determining that determines existence of the compensatory movement in a first motion based on whether or not the still state or the moving state of a first measurement body region group of the exerciser in the first motion satisfies a first criterion; and/or
second determining that determines existence of the compensatory movement in a second motion that is of lower complicity than the first motion based on whether or not the still state or the moving state of a second measurement body region group of the exerciser in the second motion satisfies a second criterion; and
identifying at least one or more body region to be the source for causing the compensatory movement based on whether or not the still state or the moving state of a third measurement body region group of the exerciser in a third motion that is of lower complicity than the second motion satisfies a third criterion, when determined in the first determining and/or the second determining that there is the compensatory movement.

18. A source body region identification system for executing a compensatory movement source body region identification method that identifies a body region to be a source for causing a compensatory movement based on a still state or a moving state in a prescribed body region of an exerciser, wherein:
the compensatory movement source body region identification method comprises:
first determining that determines existence of the compensatory movement in a first motion based on whether or not the still state or the moving state of a first measurement body region group of the exerciser in the first motion satisfies a first criterion and/or
second determining that determines existence of the compensatory movement in a second motion that is of lower complicity than the first motion based on whether or not the still state or the moving state of a second measurement body region group of the exerciser in the second motion satisfies a second criterion, and
identifying at least one or more body region to be the source for causing the compensatory movement based on whether or not the still state or the moving state of a third measurement body region group of the exerciser in a third motion that is of lower complicity than the second motion satisfies a third criterion, when determined in the first determining and/or the second determining that there is the compensatory movement; and
the source body region identification system comprises
a first recording medium recording the first criterion for determining existence of the compensatory movement in the first motion for the first measurement body region group of each exerciser and/or
a second recording medium recording the second criterion for determining existence of the compensatory movement in the second motion for the second measurement body region group of each exerciser, and
a third recording medium recording the third criterion for identifying the body region to be the source for causing the compensatory movement based on the still state or the moving state in the third measurement body group of the exerciser.
